(19) United States
(12) Patent Application Publication
(10) Pub. No.: US 2006/0247477 A1
(43) Pub. Date: Nov. 2, 2006

(54) Essential oil with citronellol and rose oxides from Dracocephalum heterophyllum benth and a process thereof

(75) Inventors: Vijay Kumar Kaul, Himachal Pradesh (IN); Umar Mahmood, Himachal Pradesh (IN); Paramvir Singh Ahuja, Himachal Pradesh (IN); Brij Lal, Himachal Pradesh (IN); Hans Raj Negi, Himachal Pradesh (IN); Virendra Singh, Himachal Pradesh (IN); Nartan Devi, (US)

Correspondence Address: FOLEY AND LARDNER LLP;SUITE 500, 3000 K STREET NW, WASHINGTON, DC, 20007, US

(73) Assignee: COUNCIL OF SCIENTIFIC AND INDUSTRIAL RESEARCH

(21) Appl. No.: 11/255,175

(22) Filed: Oct. 21, 2005

Related U.S. Application Data
(63) Continuation of application No. 10/508,995.

(51) Int. Cl. C07C 33/28

(52) U.S. Cl. 568/813

(57) ABSTRACT

This invention relates essential oil constituents citronellol and rose oxides and other constituents from a cold desert plant i.e. Dracocephalum heterophyllum benth, the present invention also provides a process for the extraction of the essential oil.

### TECHNICAL FIELD

The present invention relates to an essential oil with citronellol and rose oxides in high yield and other perfumery compounds obtained from a cold desert plant source Dracocephalum heterophyllum benth. The present invention also relates to a process for the extraction of essential oil from the plant source.

### BACKGROUND ART

The generic name Dracocephalum Linn is derived from Greek words Drakon meaning dragon and Kephale meaning head referring to the appearance of the heads of the flowers. The genus Dracocephalum comprises about 50 species distributed in Northern Hemisphere i.e. Southern Europe, North America, North Africa and temperate Asia Reference may be made to Hooker, J. D. Flora of British India, 1872-97 vol-4, 666, London.

Another reference may be made to Bailey, L. H. 1976. Hortus Third (revised edition), MacMillan Co. N.Y. 398 D. heterophyllum is a native to western Himalaya and Tibet Reference may be made to Hay, T. 1937. Gard. Chron. 101:203. It is one of the 8 species known so far from Indian subcontinent. D. heterophyllum has been reported from different parts of India including J & K, H. P., Uttaranchal and Sikkim Himalaya between the elevation of 3000-5200 m.

Reference may be made to Hooker, J. D. Flora of British India, 1872-97 vol.-4, 666, London, another reference may be made to Anon., 1952, vol.-III, PID, New Delhi, yet another reference may be made to Hajra, P. K. and Balodi, Vipin 1995, Plant Wealth of Nanda Devi Biosphere Reserve BSI, Dehra Dun pp. 277. This plant has not yet been explained for any commercial utility. But on the basis of recent field studies it has been revealed that the crude extract of the plant is used in treating eye ailments like redness of eye, irritation and conjunctivitis of the native people of Spiti valley, BP. The same use has been reported earlier from Ladakh region of J&K reference may be made to Srivastava, T. N. and Gupta, O. P. 1982 in C. K. Atal and B. N. Kapur (eds.): Cultivation and utilization of medicinal plants PP-519. RRL Jammu.

A literature survey on Dracocephalum shows that the essential oil of many species have been reported. Reference may be made to Ahmedi, L., Mirza, M. (2001). Volatile constituents of Dracocephallum aucheri Boiss. J. Essent. Oil Res., 13, 202. The result shows that there are remarkable differences in the major constituents.

On the basis of major chemical constituents of Dracocephalum species, it can be divided into 5 major chemotypes

1) citral, geraniol type

2) p-mentha-1,8-diene-1-ol, limonene type

3) 1,8-cineole, limonene, p-cymene type

4) Sabinene, germacene type and

5) Pinocamphone, b-pinene type.

Reference may be made to Misra, L. N., Shawl, A. S. Raina, and V. K. (1988) Volatile constituents of Dracocephalum nutans. Planta Med. 53, 165.

Keeping in view the significant chemical diversities in essential oil this genus, a detail study of the essential oil of D.heterophyllum is undertaken. However, some of the chemical constituents of the essential oils of this plant have been reported recently. Reference may be made to Lu-Man, Tian-Xuan, Lu-M, Tian-X, (1999), Analysis of essential oil of D. heterophyllum 34, 925.

Recently, the plant material (whole plant) of D. heterophyllum has been collected from nature i.e. Shagtal-Gete (4400-4500m) and Kibber (4100-4200m) of Lahul-Spiti region in Himachal Pradesh in the month of August 2001.

The study has been carried out to analyze its essential oil constituents and it is revealed that D. heterophyllum has an interesting chemo-type containing highest content of citronellol and rose oxides. These yields are substantially higher than any other reported species of Dracocephalum and is designated 6th type of chemo-type and named it as citronellol, rose oxide type.

Acclimatization process of this plant in ex-situ conditions i.e. in the experimental farm (under controlled condition) of the Institute at Palampur (1300 m) has also been carried out to perform comparative studies of the essential oil this plant

In order to collect ethnobotanical, ecological and floristic field data from higher altitudes of western Himalaya, a field survey was conducted in Spiti valley of Lahul-Spiti district of Himachal Pradesh, India in the month of August 2001.

While conducting field surveys in the said area, some patches of D. heterophyllum Benth. were located near by Gete (4400-4500 m) and Kibber (4100-4200 m) Villages in Spiti valley. Population sampling of the same was carried out and simultaneously plant material (whole plant) was collected for chemical examination. The voucher specimens of the plant (containing field numbers 1583-87, 1895 & 1903) were collected, processed, identified and deposited in the herbarium of the Institute (PLP).

The authenticity of the species was confirmed by way of matching with the specimens documented in the herbarium of Northern Circle, Botanical Survey of India (BSD) Dehra Dun.

### OBJECTS OF THE PRESENT INVENTION

The main object of present invention is to discover a cold desert plant as a new source of essential oil.

Another object of present invention is to identify/select D. heterophyllum as a new source of perfumery compounds of commercial significance.

Still another object of present invention is the domestication and cultivation of this plant for essential oil production.

Further object of present invention is to conduct the comparative studies of the essential oils of both wild and cultivated populations of D. heterophyllum so as to select the elite clones.

Still another object of present invention is to discover a high yielding natural source of citronellol and rose oxides.

Yet another object of present invention is to harness the potential of this natural plant resource for the economic benefits of the native people living in high mountains.

### SUMMARY OF THE INVENTION

The present invention provides an essential oil with citronellol and rose oxides in high yield and other perfumery compounds obtained from a cold desert plant source Dracocephalum heterophyllum benth. The present invention also relates to a process for the extraction of essential oil from the plant source.

### DETAILED DESCRIPTION OF THE INVENTION

Accordingly, the present invention provides an essential oil obtained from natural and cultivated cold desert plant Dracocephalum heterophyllum Benth having high value of perfumery compounds, said essential oil obtained from:
(i) a natural plant comprising: cis-rose oxide 1.6%, trans-rose oxide 0.5%, citronellal 6.7%, citronellol 74.9%, geranial 1.5%, citronellyl acetate 6.7%, neryl acetate 0.7%, geranyl acetate 1.3%, spathulenol 1.5%, citronellyl-isobutyrate 0.8%, citronehol formate 0.2% and α-bourbonene 0.4%.
(ii) a cultivated plant comprising: benzaldehyde 0.2%, 6-methylheptanone 0.2%, α-pinene 0.5%, β-pinene 0.2%, linalool 0.8%, cis-rose oxide 0.6%, trans-rose oxide 0.3%, citronellal 2.5%, citronellol 54.3%, neral 1.2%, geranial 2.4%, geraniol 1.9%, citronellyl acetate 21.6%, neryl acetate 0.4%, geranyl acetate 11.7%, β-farnesene 0.1%, δ-elemene 0.5%, spathulenol 0.2% and citronellyl-isobutyrate 0.3%

An embodiment of the present invention, wherein the constituents of said essential oil are identified by Gas Chromatography (GC) and Gas Chromatography Mass Spectra (GCMS).

Another embodiment of the present invention, wherein said oil content is a new commercial source for citronellol.

Yet another embodiment of the present invention, wherein said oil content is a new commercial source for cis and trans rose oxides.

Still another embodiment of the present invention, wherein said oil content is a new commercial source for citronellyl acetate, geranyl acetate and citronellyl iso-butyrate.

Yet another embodiment of the present invention, wherein the yields of citronellol and rose oxide thus obtained are substantially higher than from any other Dracocephalum species.

Still another embodiment of the present invention, wherein the chemo-type containing highest content of citronellol and rose oxides is designated as 6th type of chemo-type and named as citronellol, rose oxide type.

Further embodiment of the present invention, wherein the essential oil yield from D. heterophyllum is about 0.45% on fresh wt. basis.

The present invention also provides a process for the extraction of essential from a new plant source, Dracocephalum heterophyllum Benth, said process comprising the steps of:
(a) charging plant material with water in a round bottom flask attached to Clevenger type apparatus;
(b) heating the plant material to a boiling temperature;
(c) condensing the vapor to separate the volatile oil from the upper layer of distillate to obtain the essential oil;

An embodiment of the present invention, a process wherein the essential oil yield from D. heterophyllum is about 0.45% on fresh wt. basis.

Yet another embodiment of the present invention, a process wherein the plant material is selected from the whole plant.

Still another embodiment of the present invention, a process wherein the plant material is used obtained both from high altitude natural plants and from low altitude cultivated plants.

Further embodiment of the present invention, wherein D. heterophyllum is cultivated in the experimental farm (under controlled condition) of the Institute at Palampur (13300 m) and essential oil is distilled on Clevenger apparatus by hydrodistillation yield 0.4% on fresh wt. basis.

Yet another embodiment of present invention, wherein the GCMS of the essential oils was carried out on Shimadzu instrument using CP Sil 8CB, non-polar column (5% phenyl polysiloxane), column length 30 mts (i.d. 0.25 mm) carrier gas helium, temperature programmed from 100° C.-250° C. at the rate of 6° C./min.

### EXAMPLE

Method of Extraction of Essential Oil

1 kg of fresh plant material was charged along with water in 1:2 ratio in a 5 litre round bottom flash This flash is attached to a Clevenger type apparatus and the whole system is put on heating mental and heated. After few minutes the material inside start boiling. The vapour so formed is condensed through condenser in Clevenger type apparatus. The condensed distillate get collected in a measuring tube, this process is continued for 3 h and the volatile oil is separated from upper layer from the distillate because of its density difference, as oil is lighter than water and the quantity of oil is measured.

Advantages

The present invention will open new vistas in R&D leading to harness the potential of D. heterophyllum at commercial level by identifying an alternate promising source of two isomers cis and trans-rose oxides, the highly significant perfumery compounds in addition to rose and geranium oils.

This plant contains highest percentage of citronellol among the known sources of Dracocephalum species and can be utilized as an alternate source of this compound.

1-11. (canceled)

12. A novel essential oil mixture obtained from new chemo-type namely citronellol-rose oil type of natural and cultivated desert plant Dracocephalum heterophyllum Benth having high value of perfumery compounds, yield of about 0.45% on fresh wt. basis, said essential oil mixture obtained from:
(i) a natural plant comprising: cis-rose oxide 1.6%. trans-rose oxide 0.5%, citronellal 6.7%, citronellol 74.9%. geranail 1.5%, citronellyl acetate 6.7%, neryl acetate 0.7%, geranyl acetate 1.3%, spathulenol 1.5%, citronellyl-isobutyrate 0.8%, citronellol formate 0.2% and α-bourbonene 0.4%.
(ii) a cultivated plant comprising: benzaldehyde 0.2%, 6-methylheptanone 0.2%, α-pinene 0.5%, β-pinene 0.2%, linalool 0.8%, cis-rose oxide 0.6%, trans-rose oxide 0.3%, citronellal 2.5%, citronellol 54.3%, neral 1.2%, geranial 2.4%, geraniol 1.9%, citronellyl acetate 21.6%, neryl acetate 0.4%, geranyl acetate 11.7%, β-farnesene 0.1%, δ-elemene 0.5%, spathulenol 0.2% and citronellyl-isobutyrate 0.3%.

13. The essential oil mixture according to claim 12, wherein the constituents of said essential oil mixture are identified by Gas Chromatography (GC) and Gas Chromatography Mass Spectra (GCMS).

14. The essential oil mixture according to claim 12, wherein the yields of citronellol and rose oxide thus obtained are substantially higher than from any other Dracocephalum species.

15. The essential oil mixture according to claim 12, wherein the chemotype containing highest content of citronellol and rose oxides is designated as 6th type of chemo-type and named as citronellol, rose oxide type.

16. Use of an essential oil mixture according to claim 12, wherein said oil mixture content is a new commercial source for obtaining citronellol.

17. Use of an essential oil mixture according to claim 12, wherein said oil mixture content is a new commercial source for obtaining cis and trans rose oxides.

18. Use of an essential oil mixture according to claim 12, wherein said oil mixture content is a new commercial source for obtaining citronellyl acetate, geranyl acetate and citronellyl iso-butyrate.

19. A process of extraction of essential oil mixture from a new plant source, Dracocephalum heterophyllum Benth, said process comprising the steps of:
(a) charging plant material with water in a round bottom flask attached to Clevenger type apparatus;
(b) heating the plant material to a boiling temperature;
(c) condensing the vapor to separate the volatile oil mixture from the upper layer of distillate to obtain the essential oil mixture;

20. A process according to claim 19, wherein the essential oil mixture yield from D. heterophyllum is about 0.45% on fresh wt. basis.

21. A process according to claim 19, wherein the plant material is selected from the whole plant.

22. A process according to claim 19, wherein the plant material is used obtained both from high altitude natural plants and from low altitude cultivated plants.
